(19) United States
(12) Patent Application Publication
(10) Pub. No.: US 2004/0228756 A1
(43) Pub. Date: Nov. 18, 2004

(54) Laser air purifying, decontamimating, and sterilizing unit

(76) Inventor: Lambert Darryl Berry, Hawthorne, NV (US)

Correspondence Address: GRAYBEAL, JACKSON, HALEY LLP, 155 - 108TH AVENUE NE, SUITE 350, BELLEVUE, WA, 98004-5901, US

(21) Appl. No.: 10/640,477

(22) Filed: Aug. 11, 2003

Related U.S. Application Data
(60) Provisional application No. 60/403,326, filed on Aug. 12, 2002.

(51) Int. Cl. A61L 9/00; A62B 7/08; B01J 19/08; A61L 2/00

(52) U.S. Cl. 422/22; 422/1; 422/4; 422/5; 422/121; 422/124; 422/186; 422/300; 422/306

(57) ABSTRACT

An air purification system that uses laser beams to purify air. A laser beam is set to sweep across the interior of a box that is open at two ends to the flow of air. The laser beam is of sufficient strength to destroy or neutralize any dust particles, pollen, pathogens, allergens, or gasses that are present in the flow of air through the box. An air baffle box is utilized at each end of the box with the air flow to prevent the laser beam from escaping from the box. A second preferred embodiment of the present invention is the use of a fan laser beam to purify a flow of air. Said fan laser beam is suppled to a box through which air is flowing in such a manner that all air passing through the box must also pass through said fan laser beam. An air baffle box is utilized at each end of the box to prevent the escape of laser beams.

### CROSS-REFERENCE TO RELATED APPLICATIONS This application claims the benefit of PPA Ser. No. 60/403326

### FEDERALLY SPONSORED RESEARCH

Not Applicable

### BACKGROUND OF THE INVENTION—FIELD OF INVENTION

This invention relates to the ability of high powered lasers to destroy, neutralize, or volatilize pathogens, complex gasses, allergens, and other small complex particles, specifically to sterilize and decontaminate the air passing through an air duct.

### BACKGROUND OF THE INVENTION

The need for clean air has long been recognized in hospitals, clinics, dentist's offices, and dozens of other health oriented buildings, as well as most other building of all kinds. The need for clean air exists any where people congregate inside. Hundreds of different kinds of air filters have been invented and sold. Many kinds of air filters are available on the market at this time, however, only very small capacity units remove a large portion of the pathogens and filters that remove poisonous gasses generally are much smaller yet. The few filters that do remove viruses are extremely expensive, complex and require large amounts of electrical power to forces air through extremely fine pores. They are unusable for large buildings, being only used for clean rooms and other small room applications.

In recent years there has been research in the area of removing pathogens with special efforts to remove viruses. U.S. Pat. No. 6,589,489 is one example of the prior art in this area. This patent uses UV light to kill many pathogens that happen to get caught in specially constructed filters, and it apparently can kill some of the pathogens that might be carried in the air. The problem with this unit is its extreme complexity, the result of which is that it is very expensive to manufacture. Beyond that, some of the chemicals use in the construction of this unit are very toxic. An example is magnesium fluoride. Accidents could occur that would be serious and the disposal of used units would be an environmental problem. The maintenance of this unit would be very difficult because of the complex filters that would have to be changed often. One could not be assured that all pathogens, especially viruses would be destroyed.

Basically, the present air purifying units now commercially available and/or patented all have one or more of the following disadvantages:

(a) When removing viruses, the kind of filters that filter viruses become quickly clogged.

(B) Some are effective for small areas, but they cannot be use for larger buildings because of the high cost.

(C) Some use dangerous chemicals, such as fluorides, as part of the process thus leaving it possible to have serious accidents.

(D) Some generate ozone as a part of the air purification process and thus allows ozone leakage as the unit ages, and some make no effort to eliminate the ozone.

(E) All modern air purification units introduce filters or other mechanisms into the flow thus restricting the flow of air as the filter becomes clogged.

(F) Although there are some units that neutralize gasses, such as carbon monoxide, none actually destroy or neutralize poisonous gasses.

(G) None claim to destroy all pathogens. All admit some pathogens, especially viruses, still get through the purification unit.

### BACKGROUND OF INVENTION—OBJECTS AND ADVANTAGES

Several objects and advantages of the present invention for air purification, decontamination, and sterilization are:

(a) removes all pathogens, dust particles, allergens, gasses, such as poisonous organic gasses, and poisonous non organic gasses.

(b) does not place any filters or other mechanisms into the flow of air, thus presenting no restriction to the flow of air.

(c) uses no chemicals.

(d) lower cost than other air purifier units of the same capacity with similar attributes.

(e) in most cases this invention eliminates the need for air filters as all particles are volatilized and the remaining gasses are mostly reduced to the non poisons elemental stage.

(f) volatilization of all particles is instantaneous which is less time dependent than, for example, ultra violet radiation which can take up to 4 days to kill pathogens imbedded in a filter.

Further objects and advantages are to provide clean pure sterilized air to hospitals, clinics, air lines, subways, movie theaters and thousands of other buildings where people congregate. This invention can be use to neutralize biological and neurological attacks by terrorists in military installations and civilian buildings of all kinds thus minimizing the effect such attacks could have. Any air from the outside would have all biological agents or neurological agents destroyed or neutralized as it was passed through the Laser Air Purifier unit which would be built into the air conditioning system of the building. Any such agents that might be released inside a building would only have an effect in the room that they were released as all air in such buildings is drawn into the air-conditioning system from each room individually and thus would go through the Laser Air Purifier Unit before being routed to other rooms.

A further object and advantage of the present invention would be to pass the exhaust gasses from combustion engines through the Laser Air Purifier Unit wherein the carbon monoxide gas and other complex gasses from the combustion would be destroyed thus creating purified air and water. Still further objects and advantages will become apparent from a consideration of the ensuing description and drawings.

### SUMMARY

In accordance with the present invention there is provided a Laser Air Purifying, Decontaminating, and Sterilizing Unit,” whereby a section of an air duct is engineered to be coated inside with highly reflective material similar to a mirror. A laser beam of high enough intensity to destroy or neutralize particles of any nature that might be in the air flowing through the air duct is caused to sweep through the section of the air duct that is coated inside with the mirror reflective material. The laser beam sweeps through the air duct section from one side to the other and is reflected back and forth within the said air duct thousands of times causing walls of laser energy within said air duct in such a manner that all air passing through the air duct section must come in contact with said laser beam. Thus any dust, pollen, pathogens, allergens, and gasses are destroyed or neutralized by said laser beam. Baffles of material that are absorptive to laser beams are arrange at each end of the section of air duct in which said laser beam is employed so that the air is not obstructed, but the said laser beam is prevented form passing out of the air duct section.

According, a second preferred embodiment of the invention, there is provided a Laser Air Purifying, Decontaminating, and Sterilizing Unit,” whereby a fan laser beam is directed into a section of an air duct that is engineered to be coated inside with highly reflective material similar to a mirror. Said fan laser beam is slightly tilted so that it reflects back and forth down the interior of said section of air duct. Because said fan laser beam fans out in an ever widening path, within several reflections, walls of laser beams will exist so that no air can be passed through each wall without totally contacting said laser beam. Accordingly, anything contained within the air flowing through the air duct section will be destroyed or neutralized by the laser beam. Thus, any dust, pollen, pathogens, allergens, and gasses are destroyed or neutralized by the laser beam. Baffles of material that are absorptive to laser beams are arrange at each end of said section of air duct in which said fan laser beam is employed so that the air is not obstructed, but the said fan laser beam is prevented form passing out of the air duct section.

### DRAWINGS—FIGURES

In the drawings, FIG. 1 shows the various aspects of a d embodiment of the present invention.

FIG. 2 shows a second preferred embodiment of the present invention.

FIG. 3 shows a baffle arrangement used to prevent the Laser beam from escaping from an air purifier box in which the lasers of both embodiments of the present invention are used.

### DRAWINGS—REFERENCE NUMERALS

12 electric motor

14 mirror surfaces

16 rotor with plurality of flat mirror surfaces

18 motor shaft

20 opening

22 laser unit

24 laser beam

28 air purification box

32 mirror surfaces

34 fan laser unit

36 opening

38 fan laser beam

40 air purification box

42 air baffle

44 air baffle

46 air baffle

48 air baffle

50 air baffle

52 air baffle box

### DETAILED DESCRIPTION—FIG. 1 PREFERRED EMBODIMENT

FIG. 1 shows a laser air purification section that can be installed in any air duct or air flow system for the purposes of air purification, decontamination, and sterilization. A laser unit 22 that generates a laser beam 24 powerful enough to destroy or neutralize dust particles, pollen, pathogens, allergens, and gasses is mounted by mechanical means so that laser beam 24 that emanates from the laser unit 22 can strike a mirror surface 14 mounted on a rotor with plurality of flat mirror surfaces 16 in such a way that the laser beam will be reflected into an air purification box 28 through an opening 20 in the air purification box 28. The rotor with plurality of flat mirror surfaces 16 is mounted on a motor shaft 18 which is spun by an electric motor 12. The interior of the air purification box 28 is covered with mirror surfaces 32. Inside the air purification box 28 a single laser beam 24 is shown for clarity purposes to reflect only two times. In actuality, the laser beam 24 will reflect thousands of times from side to side from the mirror surfaces 32.

The rotor with plurality of flat mirror surfaces 16 consists of 6 flat surfaces on which is mounted 6 reflecting mirror surfaces 14. The air purification box 28 is a box like section that can be constructed to the size of any air duct. It is lined in the interior with mirror surfaces 32 and has an opening 20 designed especially to permit the entrance of the laser beam 24. The mirror surfaces 14 and 32 are made of glass mirrors or of highly polished metal surfaces, such as sliver or gold, or other special alloys or other plastic reflective surfaces. In the preferred embodiment the mirror surfaces 14 and 32 would be made of highly polished metal as that would not require that the laser first pass through the glass of a mirror.

### Operation—FIG. 1

The laser air purification section purifies air as it flows through the air purification box. The laser unit 22 emanates the laser beam 24 which reflects from the mirror surfaces 14 as the motor spins the rotor with plurality of flat mirror surfaces 16. The laser unit 22 and the rotor with plurality of flat mirror surfaces 16 are mounted by a mechanical means so that the laser beam 24 can only reflect into the air purification box 28.

Because the spinning of the rotor with plurality of flat mirror surfaces 16 continuously changes the angle from which the laser beam 24 reflects into the opening 20, the laser beam 24 is caused to sweep across the inside of the air purification box 28. The laser beam 24 then reflects from the various mirror surfaces 32 thousands of times filling the interior of the air purification box 28 with thousands of sweeping laser beams.

The spinning of the rotor with plurality of flat mirror surfaces 16 causes the reflecting mirror surfaces 14 to reflect a plurality of sweeping laser beams for each rotation. For instance, should the electric motor spin a hexagon rotor with flat mirror surfaces 1000 RPM, there will be 6000 sweeps of the laser beam 24 across the inside of the air purification box 28. This figure will be multiplied by the number of times the reflective mirror surfaces 32 reflects the sweeping laser beam 24 to contact all the air moving through the air purification box 28. Thus dust particles, pollen, pathogens, allergens, and gasses are contacted with the laser beam 24 and destroyed or neutralized.

The laser unit 24 should be chosen by someone skilled in the art of the use of lasers that will be proper to destroy or neutralize dust particles, pollen, pathogens, allergens, and unwanted gasses.

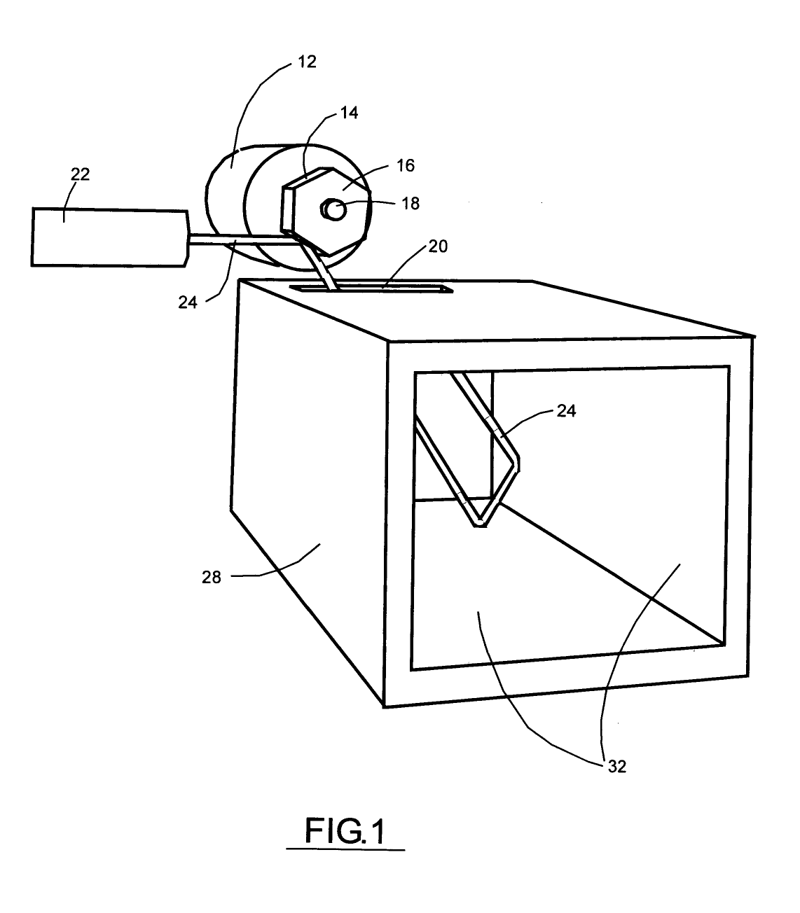

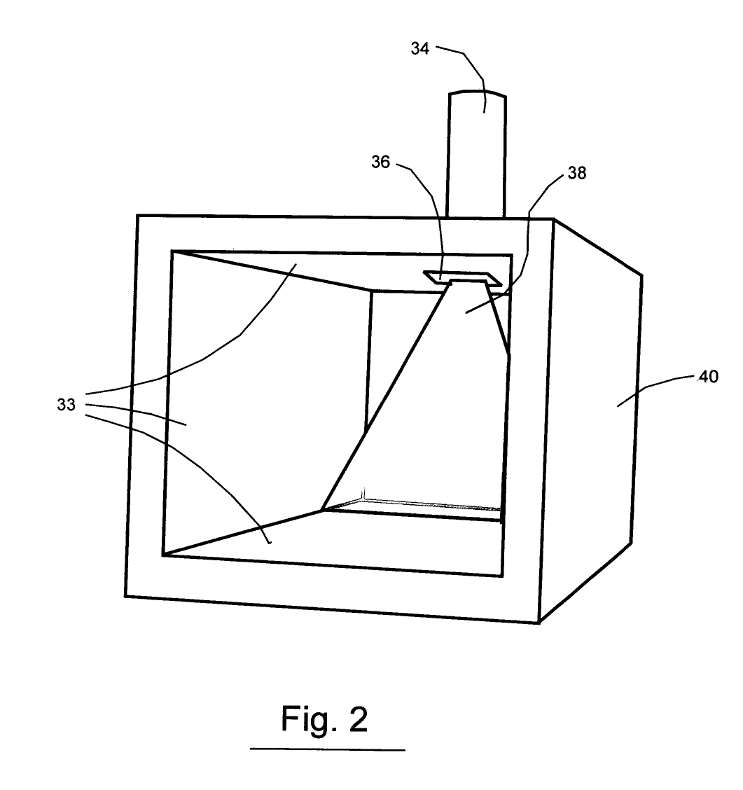

### DETAILED DESCRIPTION—FIG. 2 PREFERRED EMBODIMENT

FIG. 2 shows a fan laser air purification section that can be installed in any air duct for the purposes of air purification, decontamination, and sterilization. A fan laser unit 34 is mounted by mechanical means so that a fan laser beam 38 emanating from the fan laser unit 34 will pass through an opening 36 in an air purification box 40. The mechanical means is adjusted so that the fan laser beam enters into the air purification box 40 on a slight slant. This allows the fan laser beam 38 to reflect hundreds or thousands of times off of mirror surfaces 33 in the interior of the air purification box 40.

The fan laser unit 34 is of the type of laser that produces a laser beam that increases in width as it emanates farther and farther from the fan laser unit. Although there are several methods of creating fan laser beams at this time, any one of the known methods will work for the purposes described herein.

The air purification box is a box like section that can be constructed to the size of any air duct or air flow system. It is lined in the interior with the mirror surfaces 33 and has an opening 36 designed especially to permit the entrance of the fan laser beam 38.

### Operation—FIG. 2

The fan laser air purification section purifies air as it flows through the section. The fan laser unit 34 emanates the fan laser beam 38 which passes through the opening 36 on a slight slant so that the reflection of the fan laser beam 38 does not reflect back out of the opening, but rather, continues reflecting back and forth in the mirror surfaces 33 toward the other end of the air purification box 40 making a wall of laser energy in each reflection. Air passing through the fan laser air purification box 40 must pass through the many reflected walls of laser energy causing all of the air to be contacted with laser energy thus destroying or neutralizing dust particles, pollen, pathogens, allergens, and gasses contained in the air.

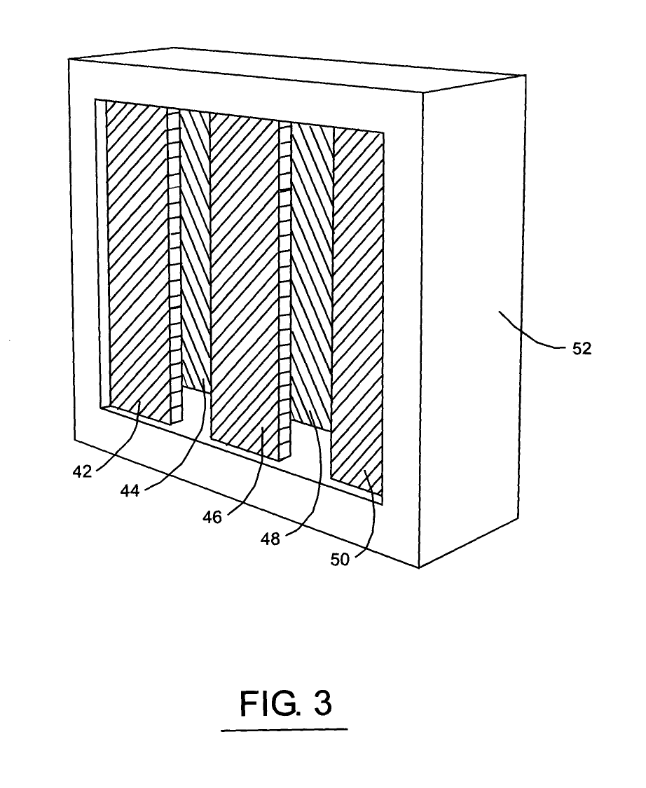

### Detailed Description—FIG. 3

FIG. 3 illustrates an air baffle laser containment section that is used to prevent the laser beam 24 and the fan laser beam 38 from escaping from the air purification boxes 28 and 40. The inside of the air baffle box 52 is coated with laser absorptive material. Air baffless 42, 44, 46, 48, and 50, also coated with laser absorptive material, are mounted within the containment box 52. Air may pass around the air baffles.

The Air baffles 42, 46, and 50 are mounted in an upright position in the front of the containment box 52, and the air baffles 44 and 48 are mounted behind the openings that are between the front air baffles.

### Operation—FIG. 3

One air baffle laser containment section shown in FIG. 3 is attached to each end of the laser air purification sections shown in FIGS. 1 and 2 when used to purify air. The air baffle containment section prevents the laser beam 24 and the fan laser beam 38 from escaping from the laser air purification sections. The air baffles 42, 44, 46, 48, and 50 are arranged in the air baffle box 52 so that there are no straight through shots that would allow a stray laser beam to exit the air purification section. The laser absorptive material coating the air baffles prevents any further reflection of the laser beams, thus effectively stopping the laser beams before they can exit. Although a particular air baffle pattern is shown, many different air baffle patterns could be used without departing from the basic concept to prevent the escape of laser beams.

### Advantages

From the description given above, a number of advantages of my laser air purification unit become evident:

(a) The laser air purification unit can sterilize air of all dust, allergens, pathogens, and poisonous gasses making the air much purer than standard filters.

(b) The laser does not offer any resistance to the flow of air and uses much less power than other air purification systems that do the same job.

(c) The cost of the laser air purification systems is low enough that it is practical for large buildings and public places.

(d) Destruction of pathogens is always instantaneous.

(e) No chemicals of any kind are used.

(f) The use of the laser air purification unit in hospitals could prevent cross infections from occurring.

(g) Because the laser air purification unit destroys or neutralizes pathogens in the air it increases the safety of buildings using the unit from various kinds of terrorist attacks.

### Conclusions, Ramifications, and Scope of Invention

From the above descriptions the reader can see that my laser air purification unit has many advantages over non laser purification units. While the above description contains many specified points, these should not be construed as limitations on the scope of the invention, but rather as an exemplification of one preferred embodiment thereof. Many other variations are possible. For example, FIGS. 1 and 2 show square or rectangular the air purification boxes 28 and 40. In another embodiment, the air purification box can have another shape, such as a round like a air pipe, or a hexagon shape or other such shapes as long as the laser beam can be made to contact all of the air passing through.

The descriptions show a method whereby the laser beam 24 is caused to sweep across the interior of the air purification box 28 as it is reflected from the mirror surfaces 14 spinning on the rotor with plurality of flat mirror surfaces 16. There are, however, a number of different methods of causing a laser beams to sweep including several electronic methods. Providing that the laser beam does sweep, any method of sweep is acceptable to the present invention and such other sweep methods do not depart from the spirit of the method of the present invention.

In yet another embodiment of the laser air purification unit a blower or fan can be attached to one end of the purification unit to force air through the unit.

A scope of the present invention is the improvement of all air purification systems. Another scope of the present invention is to use it to destroy the carbon monoxide gasses from the exhaust of a combustion engine by passing the exhaust through the laser air purification unit.

I claim:

1. A method of purifying air, comprising:
(a) a laser unit that generates a laser beam that can destroy or neutralize dust particles, pollen, pathogens, allergens, gasses, or any other contaminate that might be present in the air, and
(b) means whereby said laser beam is contained within an enclosure and means whereby said laser beam is caused to sweep across the inside of the enclosure in such a manner that all the air passing through said enclosure contacts the sweeping laser beam, and
(c) means whereby said laser beam is prevented from exiting from the area through which the air to be purified is passed.

2. A method of purifying air, comprising:
(a) a laser unit that generates a fan laser beam, that continuously expands as it moves away from the laser unit, with such power that it can destroy or neutralize dust particles, pollen, pathogens, allergens, gasses, or any other contaminate when it is contained within an enclosure, and
(b) means whereby said fan laser beam is contained within an enclosure through which the air to be purified is passed in such a manner that all the air must pass through said fan laser beam, and
(c) means whereby said fan laser beam is prevented from exiting from said enclosure.
